(19) United States
(12) Patent Application Publication
(10) Pub. No.: US 2008/0145921 A1
(43) Pub. Date: Jun. 19, 2008

(54) Tissue Bath System With Air Heating

(75) Inventor: John Pedersen, Risskov (DK)

Correspondence Address: ROBERTS, MLOTKOWSKI & HOBBES, P. O. BOX 10064, MCLEAN, VA, 22102-8064, US

(73) Assignee: DANISH MYO TECHNOLOGY A/S, Aarhus (DK)

(21) Appl. No.: 10/573,876

(22) PCT Filed: Sep. 29, 2003
(86) PCT No.: PCT/DK03/00640

§ 371 Date: Nov. 29, 2007

(51) Int. Cl. C12M 1/36; C12M 1/38

(52) U.S. Cl. 435/286.5; 435/286.1; 435/286.6

(57) ABSTRACT

A tissue bath system with containers and sample holders for holding tissue sample inside tissue medium in the containers, where each sample holder is connected to a force transducer for measuring force exerted on the holder by contraction of the tissue sample upon stimulation. The system further comprises a temperature regulation system for regulating the temperature of the tissue medium in the sample container. The system comprises a container station with chambers for placement of at sample containers in the chambers, wherein the temperature regulation system has means for providing a temperature regulated air stream into the chambers for regulation of the temperature in the container.

### FIELD OF THE INVENTION

The present invention relates to a tissue bath system for in-vitro investigation of mammal tissue, preferable investigation of contraction forces of ventricular tissue.

### BACKGROUND OF THE INVENTION

For experimental investigation as well as for clinical tests of mammal tissue, for example muscle fibres or ventricular samples, a number of apparatuses exist which comprise a number of glass containers mounted on a rack with corresponding sensors in the containers for investigations of different kind.

Such investigations are, for example, measurements of muscle contraction or contraction of ventricular tissue by force transducers in dependence of extern stimuli. Apparatuses of this kind are known from companies as World Precision Instruments Inc. (WPI) and AD Instruments and may be found in their catalogues or on their Web-pages www.wpiinc.com and www.adinstruments.com.

The tissue containers in these systems have a double wall, wherein the tissue of interest is located in the sample reservoir inside the inner wall, and wherein water from a temperature control system with circulating water is flowing between the inner and the outer wall in order to create a desired temperature in the tissue bath.

These systems have a number of disadvantages. Firstly, the containers with double walls are very expensive to manufacture. As usually a number of these containers are used, the costs for such a system are rather high. Secondly, due to the attachment to water circulation tubes between the containers, demount and mounting of the containers are difficult and time consuming, usually resulting in a very rare cleaning of the water circulation system. The rare cleaning is also due to the fact that, during the de-mounting process, water will run uncontrolled out of the system, which in many cases is experienced as an annoying moment. The consequence of the rare cleaning is that the water through the double wall system often results, on the one hand, in deposits as calcification or algae in the containers between the two walls and, on the other hand, in a general calcification of the tubing system connecting the containers for the water circulation. Thus, after already a short while of usage, the containers are practically opaque. Furthermore, the calcification implies that tubes for the heating water are very hard to remove from the containers with a risk of breaking the containers when trying to remove the tubes. This, in addition reduces the tendency to clean the system.

For such systems, it is generally known, that they already after a short while transform from a clean and neat system to a system filled with deposits of various kinds.

### DESCRIPTION/SUMMARY OF THE INVENTION

It is therefore the object of the invention to provide an improved tissue bath system, in particular for myocardial studies, which is easy to clean and does not have the aforementioned disadvantages.

This object is achieved by a tissue bath system with at least one sample container and at least one sample holder for holding at least one tissue sample inside a tissue medium in the at least one container, the at least one sample holder being connected to at least one force transducer for measuring force exerted on the at least one holder by contraction of the at least one tissue sample upon stimulation. The system further comprises a temperature regulation system for regulating the temperature of the tissue medium in the at least one sample container. The invention is further peculiar in that the system comprises a container station with at least one chamber for placement of at least one sample container in the at least one chamber, wherein the temperature regulation system has means for providing a temperature regulated air stream into the at least one chamber for regulation of the temperature in the container.

Because of the fact that the temperature regulating medium is air, no calcification occurs, and the system stays clean. Also, occurrence of algae in the tube system and in any double walls of containers is prevented. Therefore, in contrast to systems according to prior art, transparent containers in a system according to the invention stay transparent without any need of thorough and frequent cleaning of double wall systems. In addition, when cleaning is desired, no water will pour uncontrolled out of the system. Furthermore, the system is easier to keep clean and a higher standard of cleanness can be achieved because the containers can be made of a simple kind which is easy to demount and suited for an autoclave.

Heating or cooling with air has proved to be possible with high accuracy for the temperature in the reservoirs which usually are filled with a buffer solution.

Each of the at least one chamber may have an air inlet configured to regulate the flow of air through the air inlet, for example by closing or opening for the flow of air or optionally by various regulation of flow.

In a further embodiment, the tissue bath system according to the invention comprises a plurality of chambers, each chamber being configured for placement of one container. In this case, each chamber, at least partly surrounding the container, may have its own air supply, for example with a regulated air inlet which automatically only allows air for cooling or heating to enter the chamber if a container is placed in the chamber.

In a further embodiment, each sample container has a sample reservoir and a fluid channel from the reservoir traversing a wall of the container. The container station comprises a plurality of placement sections configured for placement of a sample container in each placement section, wherein each placement section has a fluid transport unit configured for coupling to the fluid channel of the container for transport of fluid through the fluid channel when the container is placed in the placement section. This embodiment is advantageous, as fluid is transported only when a container is placed in the placement section. This prevents accidents, where sample medium is spilled under uncontrolled conditions.

In a further embodiment of the invention, the fluid channel is in the lower part of a side wall of the container or in the bottom of the container. The fluid channel may be used for transport of liquid to and from the reservoir of the container and/or for transport of gases. In this case, the fluid transport unit comprise a liquid discharge/supply tube for regulated discharge or supply of solution from or to the reservoir and/or a gas supply for regulated supply of gas to the reservoir. Especially for the gas supply, it is desired that the gas is supplied from the bottom in order to have a maximum uptake of the gas in the solution in the reservoir. In order to have a thorough and even distribution of gas, for example oxygen, in the reservoir liquid, the gas supply may comprise a porous rod, for example a sintered metal rod with suitable porosity for passage of the gas through the rod extending into a connected reservoir.

In order to prevent accidents, where solution from the reservoir in the containers flows uncontrolled over the top edge of the containers and spills into the chamber, each container has an overflow tube in a upper part of the reservoir and the container station has for each placement section a co-operating overflow receiver connected with a liquid discharge system for discharge of liquid from the reservoir through the overflow tube.

In a preferred embodiment, the container has a cylindrical fluid channel extending from the container, and the fluid transport unit comprises a corresponding adapter for sealing engagement with and around the fluid channel when a container is mounted in the placement station. The fluid transport unit is demountable from the placement station for maintenance and cleaning purposes.

In a further embodiment of the invention, the container station has for each placement section a sensor configured to determine whether a container has been placed in the placement section or not. This is useful in case that the containers are used for automated experimental work in connection with data analysis. Erroneous data because of missing reservoirs are hereby avoided. Furthermore, by proper programming, it is prevented that buffer solution can be pumped through the fluid transport unit if a containner is missing I a placement section. Thus, the sensor also prevents accidents in connection with experiments that may be performed with an apparatus according to the invention.

Advantageously, the air inlet, the valves for the supply and discharge of solution and the sensor are functionally coupled to a control unit, the control unit being programmed for only allowing the transport of fluid by the fluid transport unit and only allowing the circulation of air into the at least one chamber if the sensor detects a container placed in the placement section.

The reservoirs of containers may have to be filled with a buffer solution. Such a buffer solution may be provided from a storage tank at another location, for example the other end of the laboratory. However, it is much more convenient for the user if the container station has at least one solution tank for supply of solution to at least one reservoir. The solution may be added through the top opening of the containers. However, the invention foresees that the tank is connected to a tubing system which is connected to the placement section and the fluid transport unit through a valve such that the solution may enter the reservoir through the opening in the bottom part. This is the more convenient if the reservoirs are closed with a lid.

Optionally, the reservoirs of the plurality of containers have identical heights even though other differences between the containers may be present. This does not only have an aesthetic value but also simplifies the development of technical features of the apparatus for different containers as will become more apparent below. However, typically, not only one volume of reservoirs is desired, but a range of containers with reservoirs with different volumes. For example, it may be desired to apply containers in the apparatus according to the invention with reservoir volumes of 5 ml, 10 ml, 20 ml, or 50 ml. It may even be so that small and large reservoirs are used at the same time. In this case, having regards to the fact that the containers may have a fixed heights, this may be achieved by providing at least two containers that have reservoirs with different widths in order to have different volumes.

In isolation, a container for a tissue bath system according to the invention may have a fluid channel in the bottom and an overflow tube in an upper part of the reservoir for discharge of liquid from the reservoir through the overflow tube. For an apparatus according to the invention, the possible customer may want to purchase a set of containers of the type as described in the foregoing, wherein the reservoirs of the containers have identical heights but at least two of the reservoirs have different widths in order to achieve different volumes.

A force transducer applicable for a system according to the invention may be mounted in a housing limiting the bending of the transducer to less than the proper maximum amount of bending.

### SHORT DESCRIPTION OF THE DRAWINGS

The invention will be explained in more detail with reference to the drawing, where

FIG. 1 is a drawing of the apparatus according to the invention,

FIG. 2 shows the apparatus with removed doors in front of the chambers,

FIG. 3 shows a close-up drawing of a placement section with chamber,

FIG. 4 shows a close-up drawing of a part of the front panel with tubing and valves,

FIG. 5 shows two embodiments with different volumes of the container,

FIG. 6 is a technical drawing of a placement section and a container according to the invention,

FIG. 7 shows a sample holder with force detector,

FIG. 8 an enlarged drawing of a container and the removable adapter.

### DETAILED DESCRIPTION/PREFERRED EMBODIMENT

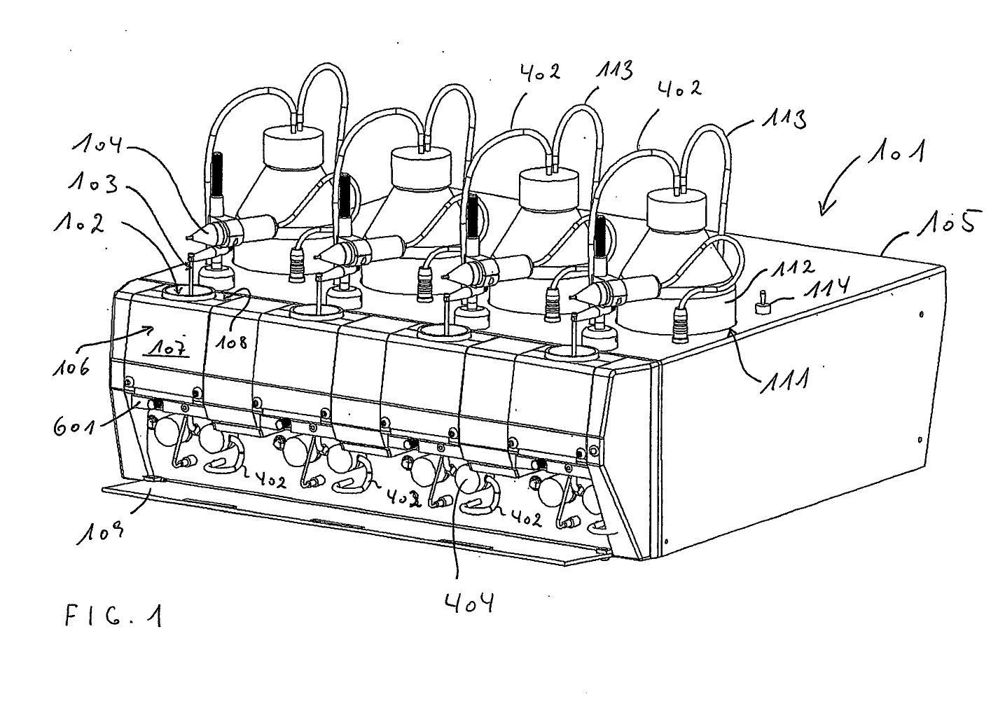

FIG. 1 is a drawing of the tissue bath system 101 according to the invention. The system 101 comprises a plurality of sample containers 102 and a plurality of samples holders 103 for holding tissue samples inside a tissue medium in the containers 102. The medium and the tissue are not shown. The sample during experiment is connected to force sensors 104 with transducers for measuring force exerted on the holders 103 by contraction of tissue samples in the container 102 upon stimulation. Such stimulation may be achieved chemically or through electrodes placed inside the medium in the container.

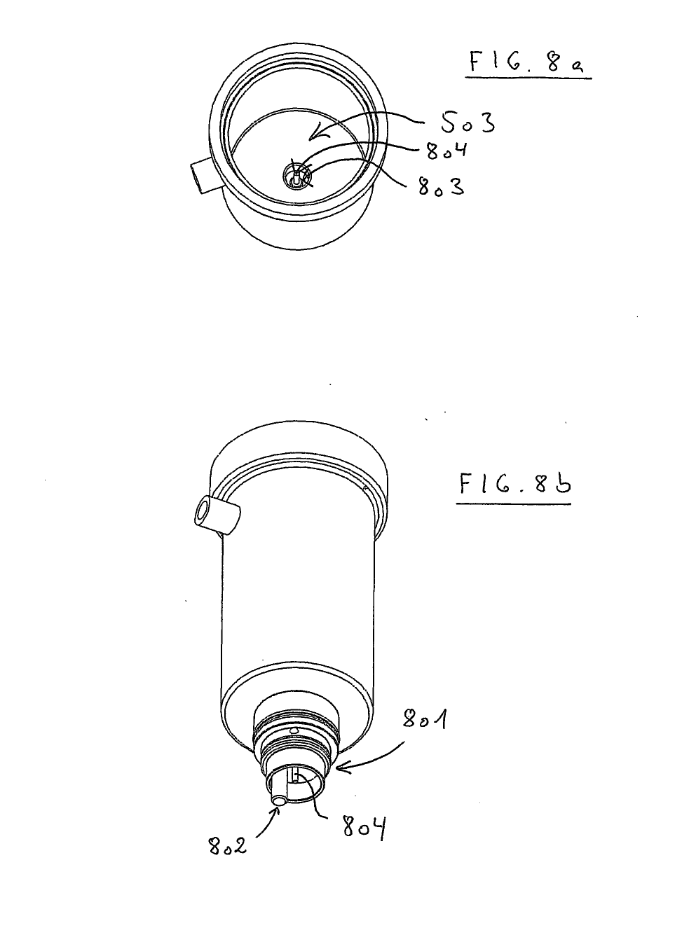

A sample holders 103 with detector is explained in greater detail in connection with FIG. 7. The sample holder 103 comprises a rod 701 with a hook 704 and a wire 702, between which the tissue 703 to be measured is mounted. Upon stimulation, the tissue 703 of interest may contract, which pulls the rod 701 and the wire 702 together, which is measured by the sensor 104 with transducer.

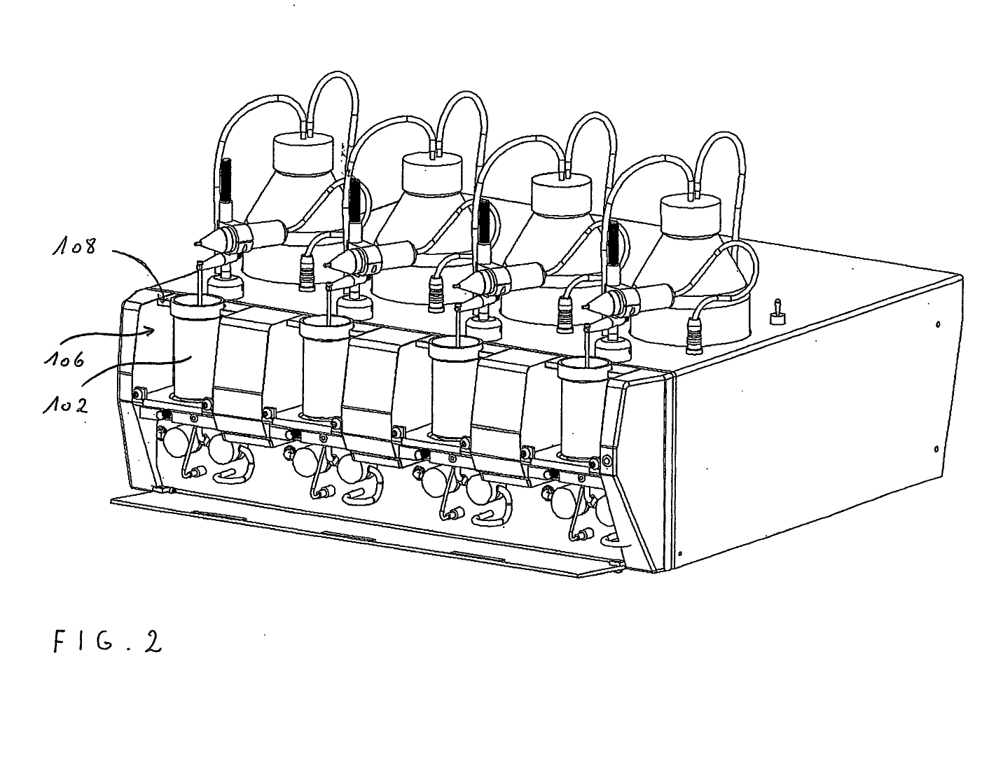

The tissue bath system 101 as shown in FIG. 1 and FIG. 2 comprises a container station 105 with a number of chambers 106 for placement of sample containers 102 in the chambers 106. FIG. 2 differs from FIG. 1 in having removed chamber doors 107. The chamber door 107, when in place, tightens the chamber 106 around the upper part of the container 102 in cooperation with a collar 108. In the shown embodiment, one container 102 is placed in each chamber 106. Even though the container station 105 may be constructed with larger chambers 106 for placement of more than one container 102, for example with only one chamber for all shown containers 102, the shown embodiment with individual chambers 106 for containers 102 is preferred, because this enhances and facilitates the control for proper temperature regulation with air. For the temperature regulation of the tissue samples and the liquid in the containers 102, the system according to the invention has a temperature regulation system for providing a temperature regulated air stream into the chamber 106 for regulation of the temperature in the containers 102.

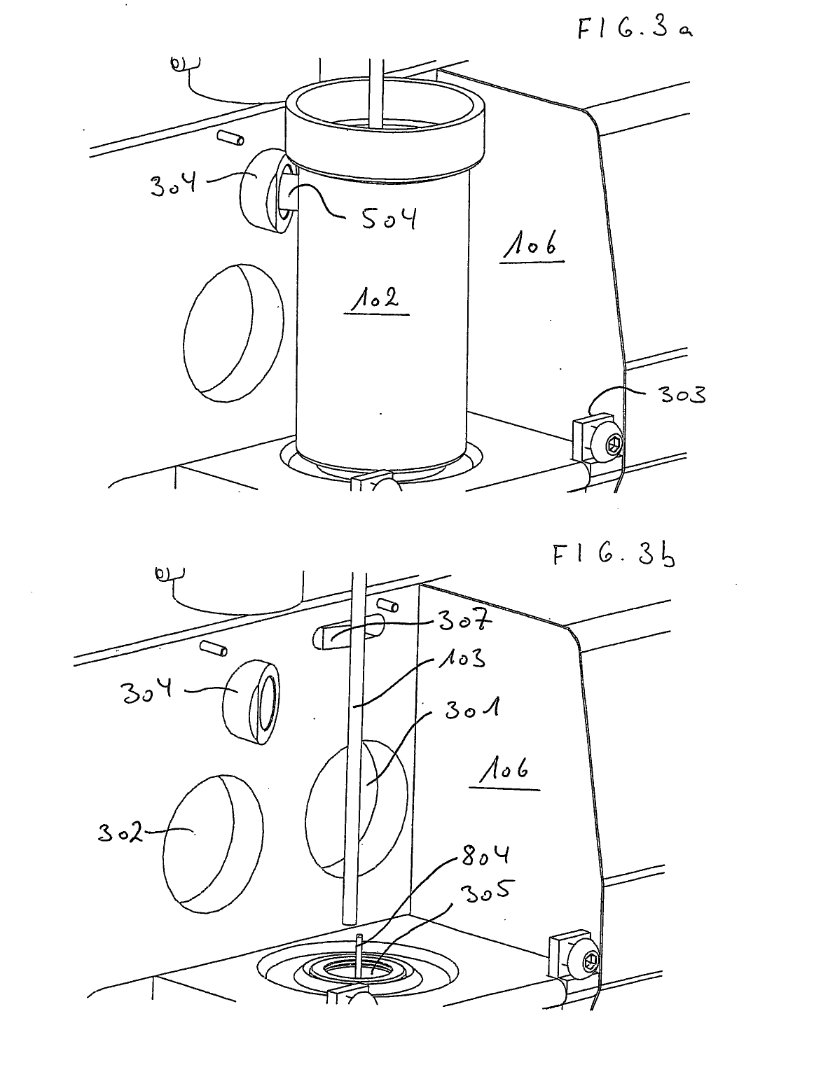

A more detailed drawing of a chamber 106 with removed chamber door 107 and collar 108 is shown in FIG. 3a with a container 102 and FIG. 3b without a container. For better understanding, the chamber door 107 as shown in FIG. 1, one side wall, and the upper collar as shown in FIG. 2 has been removed. The chamber door 107 is mounted on hinges 303, such that it can be opened in a rotational manner. The advantage of having a door 107, which can be opened, is that the chamber 107 inside easily can be cleaned and a proper access for removal and placement of the container 102 is possible.

In order to regulate the temperature of the container 102, the chamber 106 has an air inlet 310 and an air outlet 302 for circulation of air inside the chamber 106. The circulation of air inside the chamber 106 is used for regulating the temperature in the container 102. Typically, tissue samples have to be maintained at a temperature between 35 and 40 degrees centigrade, which causes a demand for heating of the containers 102 because the surroundings usually are at normal room temperature around 22-24 degrees centigrade. However, the air stream through the chamber 106 can as well be used to heat the containers 102 to any other temperature, and can optionally also be used to cool the containers if this should be necessary. The latter, however, requires that the system is constructed not only for heating but also for cooling.

The air through the inlet 301 is provided from an air heating system or an air heating/cooling system independent of the actual needs. A typical heating system would typically comprise of a fan for transporting air through the system coupled to a heated or cooled heat exchanger. A tubing system is used for transporting air from the heat exchanger to the inlet 301 of the chamber 106. Through a corresponding tubing system, the air from the outlet 302 is circulated to the heat exchanger, which is a preferred solution, but it is also possible to simply discharge the air through an outlet 302.

As it also appeared from FIG. 3, the sample holder 103 extends into the container 102. The sample holding wire 702 and the sample 703 is not shown in this figure. In order to remove the container 102, the sample holder 103 is removed from the container 102 in an upward direction. This upward direction may be caused manually or automatically. For a manual operation, a pressure on the switch 705, as shown in FIG. 7, releases the holder 103, and it may manually be lifted upwards or moved downwards in dependence on the intention. In order to ease lifting, the holder 103 may be supported with a spring forcing the holder in an upward direction. Alternatively, this upward direction may be achieved by an automatic movement of the sample holder, where the automatic movement can be initiated by a pressure on switch 705. In the automated embodiment, a simple pressure action on switch 705 causes the sample holder to be lifted to a position, for example 20 mm, above the top edge of the container 102.

A container 102 is shown in further detail in FIG. 5a The container 102 has a top part 901, the rim of which corresponds to opening defined by the collar 108 and the door 107 as shown in FIG. 1. With reference to FIG. 5a, the container 102 has a lower part 502 confining the reservoir 503 inside the container. In order to prevent the reservoir 503 to be overfilled such that buffer solution or liquid is flowing over the top part 501 of the container 102 and into the chamber 106, the container 102 has an overflow tube 504.

As shown in better detail on FIG. 3, the overflow tube 504 cooperates with an overflow receiver 304. The overflow receiver 304 cooperating with the overflow tube is connected by the liquid discharge system for discharge of liquid from the container 102 through the overflow tube 504. The discharge system may be a separate container inside the housing of the container station 105. Alternatively, the discharge system may comprise a tubing which is connected to an extern discharge unit.

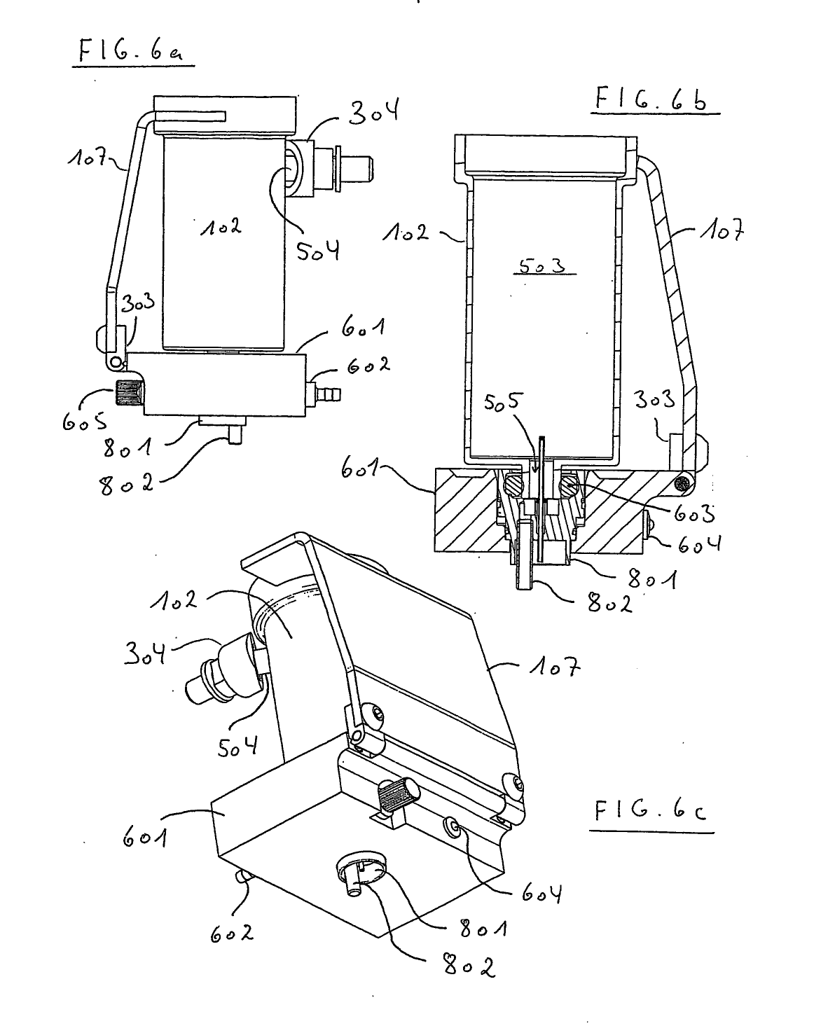

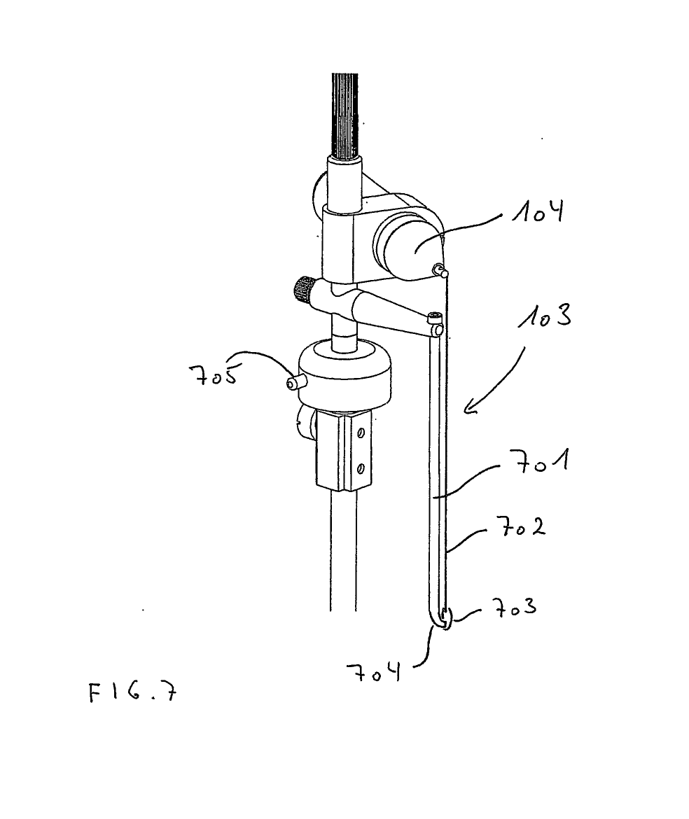

The overflow tube in co-operation with the overflow receiver may act as a support for the container 102. In this case, the container does not need a high precision for the distance from the overflow tub to the bottom of the container. Thus, as illustrated in FIG. 6b, the container need not be manufactured with such a precise length that it rests on the surface of the placement station. This is also one of the advantageous aspects that keep the production costs low for the containers.

As can be readily recognised from FIG. 5a and FIG. 5b, the container 102 has a fluid channel 505 for fluid transport in and out of the container 102. Such fluid may typically be a buffer solution in which tissue sample is stored for experimental investigation. Such fluid may be supplied or discharged through the fluid channel 505, also from the fluid channel 505, gas may be supplied, for example oxygen which is usually added to the buffer solution in order to keep the condition of the tissue sample at proper perimeters. A typical gas mixture used for living tissue consists of about 95% oxygen and 5% carbon dioxide. The relative amount of carbon dioxide influences the pH of the buffer solution.

For convenience, different containers 102 may have different volumes. However, in order for an easy mounting, the volume of the reservoirs 503 among different containers 102 may vary by varying the width of the reservoirs, which is illustrated in FIG. 5a in comparison with FIG. 5b.

The fluid channel 505 extends into an adaptor 305, as shown in FIG. 3b. The adaptor 305 may comprise an o-ring or other kinds of sealing rings for sealing engagement around the fluid channel 505. The adaptor 305 is part of the fluid transport unit 801 shown in greater detail connected to a container 102 in FIGS. 8a and 8b. The fluid transport unit 801 has a liquid discharge/supply tube 802 and a gas supply 803 for supply of gasses such as oxygen and carbon dioxide into the reservoir 503 of the container 102. The gas supply 803 surrounds a temperature sensor 804, shown enlarged in FIG. 3b, which extends into the reservoir 503. The temperature sensor 804 and the gas supply 802 as well as the liquid discharge/supply tube 802 are mounted tightly in the fluid transport unit 801 such that liquid can only be supplied and discharged through the discharge/supply tube 802 and gas can only be supplied through the gas supply 803.

With reference to FIGS. 1, 2 and 3a, if the container 102 has to be removed from chamber 305, the door 107 of the chamber 106 is opened, the sample holder 103 is removed in an upwards direction after which the container 102 can be removed by tilting it out of the discharge receiver 304 and the adaptor 305.

The fluid transport unit 801 is in contact with the solution in the reservoir 503 when a container 102 is mounted in the chamber 106 and filled with a liquid medium. Therefore, it has been foreseen that the fluid transport unit 801 also can be demounted from the container station 105 such that not only the container 102 can be cleaned in an auto clave, but also the fluid transport unit 801.

For a better understanding, a placement section 601 is shown together with a container 102 in FIGS. 6a, 6b and 6c in different perspectives and in cross section. In addition, a part of the overflow receiver 304 is shown as well in connection with the overflow tube 504 of the container 102. The placement section 601 may be a unit which can be removed from the container station 105, but this will usually not be necessary, and the placement section 601 may be secured to the container section 105, for example by corresponding screws. The placement section 601 has a gas supply connector 602 configured for cooperation with a gas supply tube in the container station 105.

In order to secure that gas and liquid only will be led through the fluid transport unit 801 if a container 102 is in place, the tissue bath system 101 according to the invention may for each placement section 601 and chamber 106 comprise a detector 307, as shown in FIG. 3b, for detecting whether a container 102 is placed in a chamber 106 or not. The detector 307 may be an infra-red detector.

In FIG. 6b, the container 102, the door 107 and the placement section 601 are shown in a cross-sectional view and, as can be readily recognised, a sealing ring 603 tightens the outer rim of the fluid channel 505 of the container 102. The fluid channel 505 is in connection with liquid discharge/supply tube 802.

In order to indicate that a container 102 is placed in a chamber 106 and has been detected by the detector 307, a small lamp 604 at the front of the placement section 601 may indicate that the container 102 has been detected and that supply and/or drainage of the fluid is possible and not blocked because of the missing signal from the detector 307.

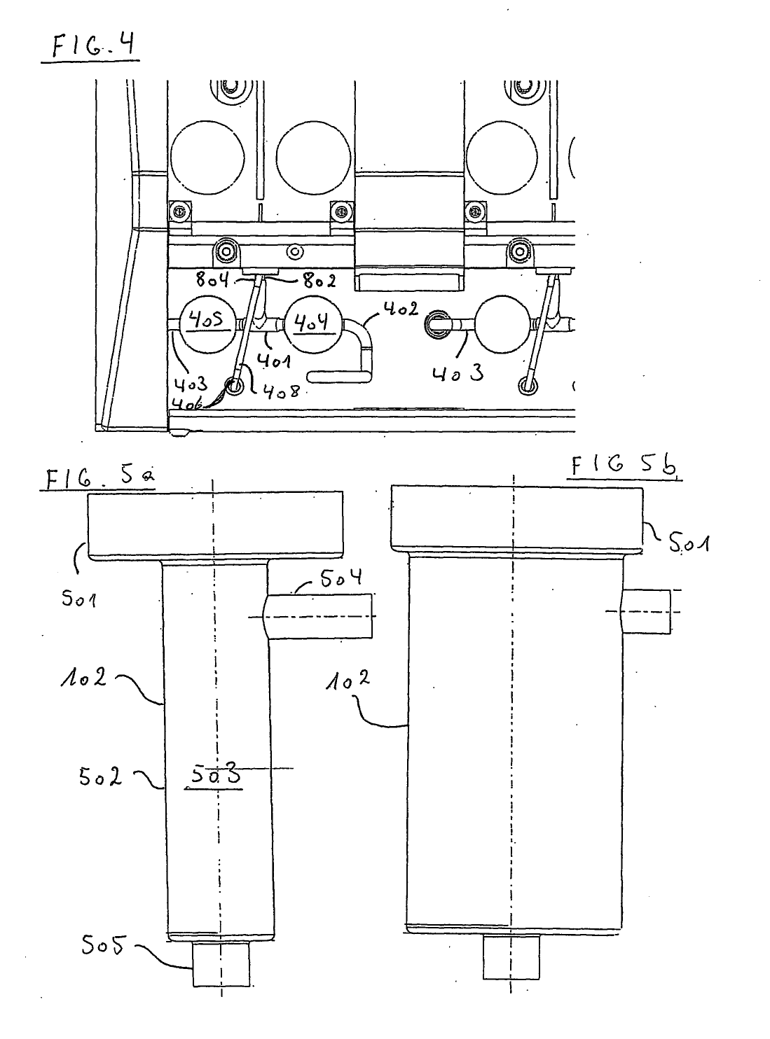

The container station 105, as shown in FIG. 1 has a front panel below the placement section inside the chamber 305. This front panel 110 may covered with a cover lid 109 where the cover lid is 109 hinged such that opening of the cover lid 109 gives access to the front panel 110. An enlarged drawing of this lower front panel is shown in FIG. 4. Comparing FIG. 6 and FIG. 4, it may readily be recognised that the discharge/supply tube 802 is connected to a T-piece 401, which again is connected to a supply tube 402 and a discharge tube 403. The amount of liquid to be supplied or discharged is regulated by valves 404 and 405, which are pressure valves exerting pressure on the elastic tubing of supply tube 402 and discharge tube 403.

The temperature sensor 804 is electrically coupled to a wire 408 having a plug 406 connected to a data read-out system. In order to remove the fluid transport unit from the chamber 106, the plug 406 as shown in FIG. 4 is pulled out the respective socket and the T-piece 40 is removed from the supply/discharge tube 802. After this simple operation, the fluid transport unit 801 can be removed from the placement section 601.

In order to regulate the gas supply, for example the supply of an oxygen mixture with carbon dioxide to the reservoir 503 in the container 102, as illustrated in FIG. 6a, the supply of gas may be regulated by a manually adjustable gas valve 605. Alternatively, the valve 605 may be automatically adjustable for example through a computer steered system (not shown). Likewise, the valves 404, 405 are electrically controlled. This may as well advantageously be achieved through a computerized control system. For flushing and cleaning of the reservoirs 503 of the containers 102, the inlet valve 404, as shown in FIG. 4, may be opened for certain periods after which the inlet valve 404 is closed and the drainage valve 405 is opened. This may be repeated for a certain number of times.

The container station, as shown in FIG. 1, has a number of receptacles 111 for containment of solution tanks 112. Each of the solution tanks 112 in the container station 105 is connected with a pressure tubing 113 to a gas storage. The pressure tubing 113 leads gas under pressure into the tank 112, whereby solution from the tank 112 is pressed into the solution supply tube 402 extending through the container station and connected to the T-piece through a regulating inlet valve 404. The gas used to press the solution out of the tank 112, is the same gas that is used for supply to the container 102, for example a mixture of oxygen and carbon dioxide. This ensures that the solution in the tank 112 has the same maximum content of gas in the solution as the solution of the container, where additional gas may be supplied. The typical gas to be used is mixture of 95% oxygen and 5% CO₂. The relative amount of CO₂ determines the PH of the buffer solution and may be varied independent of the desired PH of the solution. The solution in the tank and in the solution tank 112 will be saturated with the provided gas under usual experimental conditions.

Each of the gas supplies to the tanks 112 through pressure tubing 113 can be shut off by switches 114 in the container station 105. The gas supplies for the tanks may be kept at a constant pressure, such that the pressure defines the throughput through the T-piece in dependence of the average time by which the inlet valve 404 is open. For instance, if fast filling of the container 102 is desired, the inlet valve may intermittently be opened for longer intervals than if a slow filling of the container 102 is desired.

It is preferred that the system according to the invention is managed through a computer program from an external computer for example a PC. However for communication between the computer program and the hardware in the system according to the invention, electronic boards are located inside the container station 105. This electronic board controls all signals to and from the electronic and electric hardware components. Data from the force transducer and other detectord are transmitted through a data communication board and a data bus to the computer for analysis and further control.

The invention may be supplied by other types of sensors than explained above. For example, a pH sensor and a sensor for detection of the oxygen content may be installed in addition.

Instead of using a tubing system for transfer of heated air from a central heating station, there may be used a heat exchanger for each chamber. A small fan for air circulation may be started and stopped in dependence of the temperature in the reservoir. As a further alternative, the fan may be running continuously if a container is placed in the placement station and the heat exchanger may be activated or deactivated in dependence of the desired temperature and the temperature measured by the temperature detector.

In the shown embodiment, the fluid transport unit is used for supply and for discharge of liquid. However, a flow of buffer solution may be achieved through the container, for example by supplying liquid from the bottom and using the overflow tube. Alternatively, solution may be supplied from the top by respective supply means and the solution may be discharged through the bottom in order to achieve a steady flow.

### NUMBERING LIST

101 tissue bath system
102 sample containers
103 sample holders
104 force sensors
105 container station
106 chamber
107 chamber door
108 collar
109 cover lit
110 front panel
111 receptacles
112 tanks
113 pressure tubing
114 switch
301 air inlet
302 air outlet
303 hinges
304 overflow receiver
305 adaptor
307 detector
401 T-piece
402 supply tube
403 discharge tube
404 valve for supply tube
405 valve for discharge tube
406 tube
407 socket
408 wire
501 top part of container 102
502 lower part of container 102
503 reservoir of container 102
504 overflow tube of container 102
505 fluid channel
601 placement section
602 gas supply connector
603 sealing
604 small lamp
605 gas valve
701 rod
702 wire
703 tissue
704 hook
705 switch for movement of sample holder 103
801 fluid transport unit
802 liquid discharge/supply tube
803 gas supply
804 temperature sensor

1. A tissue bath system with at least one sample container and at least one sample holder for holding at least one tissue sample inside a tissue medium in the at least one container, the at least one sample holder being connected to at least one force transducer for measuring force exerted on the at least one holder by contraction of the at least one tissue sample upon stimulation, the system further comprising a temperature regulation system for regulating the temperature of the tissue medium in the at least one sample container, characterised in, that the system comprises a container station with at least one chamber for placement of at least one sample container in the at least one chamber, wherein the temperature regulation system has means for providing a temperature regulated air stream into the at least one chamber for regulation of the temperature in the container.

2. A tissue bath system according to claim 1 comprising a plurality of chambers, each chamber being configured for placement of one sample container in the chamber.

3. A tissue bath system according to claim 1 or 2, wherein the sample container has a sample reservoir and a fluid channel extending from the reservoir and traversing a wall of the container, wherein the container station comprises a plurality of placement sections configured for placement of a sample container in each placement section, wherein each placement section has a fluid transport unit configured for coupling to the fluid channel of the container for transport of fluid through the fluid channel when the container is placed in the placement section.

4. A tissue bath system according to claim 3, wherein the fluid channel is in the lower part of a side wall of the container or in the bottom of the container.

5. A tissue bath system according to claim 3, wherein the fluid transport unit comprise a solution supply tube for regulated supply of solution to the reservoir.

6. A tissue bath system according to claim 3, wherein the fluid transport unit comprises a gas supply for regulated supply of gas to the reservoir.

7. A tissue bath according to claim 6, wherein the gas supply comprises a porous rod configured to extend into the reservoir of a connected container and con-- figured for passage of the gas through the rod for thorough distribution of gas in the reservoir.

8. A tissue bath system according to claim 3, wherein the fluid transport unit comprises a liquid discharge tube for discharge of liquid from the reservoir.

9. A tissue bath container according to claim 2, wherein each of the at least one chamber has an air inlet that is configured to regulate the flow of air through the air inlet.

10. Tissue bath system according to claim 1, wherein the container has an overflow tube in a upper part of the reservoir and the container station has an overflow receiver configured to cooperate with such an overflow tube, the overflow receiver being connected with a liquid discharge system for discharge of liquid from the reservoir through the overflow tube.

11. Tissue bath system according to claim 3, wherein the container has a cylindrical fluid channel extending from the container, and wherein the fluid transport unit comprises a corresponding adapter for sealing engagement around the fluid channel.

12. Tissue bath system according to claim 3, wherein the container station for each placement section has a sensor configured to determine whether a container has been placed in the placement section or not.

13. Tissue bath system according to claim 12, wherein the fluid transport unit and the air inlet and the sensor are functionally coupled to a control unit, the control unit being programmed for only allowing the transport of fluid by the fluid transport unit and only allowing the circulation of air into the at least one chamber if the sensor detects a container placed in the placement section.

14. Tissue bath system according to claim 1, wherein the container station has at least one receptacle for receiving a solution tank, where the solution tank via a tubing system and a solution flow control valve is connected to the fluid transport unit.

15. Tissue bath system according to claim 1, wherein the reservoirs of a plurality of containers have identical heights but wherein at least two of the containers have reservoirs with different widths in order to have different volumes.

16. Container for a tissue bath system according to claim 1, wherein the container has a fluid channel in the bottom.

17. Container according to claim 16, wherein the container has an overflow tube in a upper part of the reservoir for discharge of liquid from the reservoir through the overflow tube.

18. A set of containers of the type as described in claim 16 or 17, wherein the reservoirs of the containers have identical heights but different widths in order to achieve different volumes.

19. A force transducer for a tissue bath system according to claim 1, wherein the transducer is mounted in a housing, the housing constructed with a mechanical bending stop limiting the bending of the transducer to less than the proper maximum amount of bending.
